(19) United States
(12) Patent Application Publication
(10) Pub. No.: US 2008/0255572 A1
(43) Pub. Date: Oct. 16, 2008

(54) Device for Following the Penetration of an Instrument in an Anatomical Structure

(76) Inventors: Reinhard Zeller, Boulogne-Billancourt (FR); Marco Brayda-Bruno, Milano (IT); Dominique Petit, Verton (FR); Gerard Vanacker, Saint-Maur (FR); Maurice Bourlion, Saint-Chamond (FR)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 11/578,009

(22) PCT Filed: Apr. 11, 2005
(86) PCT No.: PCT/FR2005/000873

§ 371 Date: Mar. 12, 2008

(30) Foreign Application Priority Data
Apr. 9, 2004 (FR) 0450721

(51) Int. Cl. A61B 17/90

(52) U.S. Cl. 606/96; 606/102

(57) ABSTRACT

A device to monitor penetration of an instrument into an anatomical structure including a voltage source supplying at least two electrodes, at least one of the electrodes carried by a part of the penetration instrument intended to penetrate the anatomical structure, the penetrating part having at least one detachable longitudinal part; and a device that measures the impedance between the electrodes or an electro-stimulator or both.

### RELATED APPLICATION

This is a §371 of International Application No. PCT/FR2005/000873, with an international filing date of Apr. 11, 2005 (WO 2005/102183 A1, published Nov. 3, 2005), which is based on French Patent Application No. 04/50721, filed Apr. 9, 2004.

### TECHNICAL FIELD

This disclosure relates to the field of spinal surgery, in particular instruments for vertebral, cervical, thoracic, lumbo-sacral or ilio-sacral drilling.

### BACKGROUND

Such devices are used to monitor drilling progress by the analysis of the electrical signal detected, whose level varies as a function of the type of biological material in contact with the electrodes placed on the instrument. When a gap is formed in the bone matter, the impedance suddenly decreases, thereby enabling the operator to better control the penetration of the drilling instrument and avoid damaging the spinal cord or the nerves by leaving the optimum path.

Such devices are in particular used for the preparation of the insertion of bone fixation screws. It is often necessary to inject a fluid or semi-fluid material in the bone cavity, for example, the bone cement intended to consolidate the bone structure. A series of interventions with distinct ancillaries and a repetition of invasive surgical procedures have to be performed.

It would be advantageous to provide a modular device to be able to carry out, after a single invasive surgical procedure, a series of surgical procedures, such as the insertion of a striated screw.

### SUMMARY

We provide a device to monitor penetration of an instrument in an anatomical structure, in particular, a bone structure, including a voltage source supplying at least two electrodes, at least one of the electrodes being carried by the part of the instrument intended to penetrate the anatomical structure, as well as a means to measure the impedance between the electrodes, or an electro-stimulator or the combination electro-stimulator/means to measure the impedance.

We also provide a device to monitor penetration of an instrument into an anatomical structure including a voltage source supplying at least two electrodes, at least one of the electrodes carried by a part of the penetration instrument intended to penetrate the anatomical structure, the penetrating part having at least one detachable longitudinal part; and a device that measures the impedance between the electrodes or an electro-stimulator or both.

### BRIEF DESCRIPTION OF THE DRAWINGS

The disclosure will be better understood when reading the following description, referring to the appended figures concerning non-limiting examples where:

FIG. 1 is a partial longitudinal section of the penetrating part of a penetration instrument forming a device;

FIG. 2 is a partial longitudinal section of the penetrating part of the penetration instrument in FIG. 1 having an operator channel; and

FIG. 3 is a partial longitudinal section of the penetrating part of the penetration instrument in FIG. 1 having a guide core.

### DETAILED DESCRIPTION

We provide a device to monitor the penetration of an instrument in an anatomical structure, in particular, a bone structure, comprising a voltage source supplying at least two electrodes, at least one of the electrodes being carried by the part of the penetration instrument intended to penetrate into the anatomical structure, as well as a means to measure the impedance between the electrodes, or an electro-stimulator or both. The part of the penetration instrument intended to penetrate into the anatomical structure has at least one detachable longitudinal part.

Therefore, depending on the location of the detachable part in the penetrating part, some of the surgical procedures following a penetration operation (insertion of a striated screw, injection of bone cement, etc.) may be carried out without another invasive surgical procedure being required other than the penetration procedure in the bone structure by the penetrating part of the instrument.

More specifically, depending on whether the detachable part forms an inner part or the lateral periphery of the penetrating part of the penetration instrument, the penetrating part is adapted to have either an operating channel or a guide core.

Therefore, according to one configuration, the penetrating part of the penetration instrument comprises an operator channel receiving the detachable longitudinal part. Withdrawal of the longitudinal part from the penetrating part leads to the formation of the operator channel. Preferably, the operator channel includes a detachable electrode.

Advantageously, the device comprises a flexible or rigid guide pin or a fluid injection tube crossing the operator channel. Advantageously, the detachable longitudinal part of the penetration instrument is central.

According to another configuration, the detachable longitudinal part forms the lateral periphery of the penetrating part of the penetration instrument. Thereby, when the longitudinal part is withdrawn from the penetrating part, the latter has a guide core. Advantageously, the guide core includes an electrode. Advantageously, the guide core is centrally located.

Advantageously, the detachable longitudinal part is an electrode.

The device is intended to monitor penetration of an instrument in an anatomical structure, and in particular in a bone structure.

The device comprises, in addition to the penetration instrument, a voltage source supplying two electrodes as well as a means to measure the impedance between said electrodes.

To provide a warning in case of variations in impedance due to the formation of a gap in the bone structure during the penetration operation with the instrument, at least one of the electrodes is carried by the part of the penetration instrument intended to penetrate in the bone part.

In the following examples, the penetrating part of the penetration instrument advantageously carries the two electrodes, thereby providing an autonomous device, not requiring any outside cables.

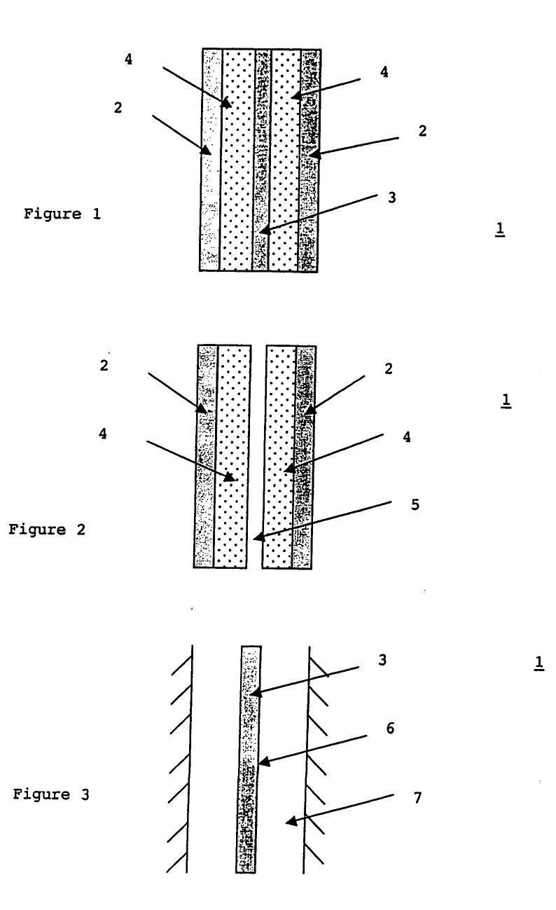

FIG. 1 represents a partial longitudinal section of the penetrating part (1) of the penetration instrument. The penetrating part (1) is formed by a rod comprising a first external electrode (2). The electrode (2) is placed laterally and on the entire periphery of the penetrating part (1).

A second electrode (3), placed in the penetrating part (1) of the penetration instrument, extends longitudinally in the former and is isolated from the first electrode (2) by an insulant (4).

The electrodes (2, 3) and the insulant (4) are, in this example, advantageously coaxial. It is understood that the scope of this description is in no way limited to such an arrangement of the electrodes (2, 3).

As illustrated by FIGS. 2 and 3, the penetrating part (1) of the penetration instrument has a detachable longitudinal part, whose withdrawal allows for the performance of surgical procedures following the drilling of the bone structure.

Advantageously, the longitudinal part removed from the penetrating part (1) of the penetration instrument may be replaced in it, so that the penetration instrument may again be used in its first function, that is, the drilling of the bone structure.

According to one aspect, the detachable longitudinal part forms an internal part of the penetrating part (1) of the penetration instrument. Advantageously, the detachable longitudinal part includes the internal electrode (3) (FIG. 2). Thus, when the internal electrode (3) is withdrawn from the penetrating part (1) of the penetration instrument, an operator channel is formed in the penetration instrument.

Due to the arrangement of the electrodes (2, 3) in the penetrating part (1) of the penetration instrument, the operator channel (5) is central.

The channel (5) then allows for different operations such as the passage of an injection piece for a fluid (bone cement, for example), the insertion of an optic fibre or an image sensor as a diagnostic tool, for example, or even the passage of a guide pin for the placement of a striated screw for bone fixation. In this case, once the guide pin is placed in the operator channel (5), the penetrating part (1) of the penetration instrument is withdrawn from the cavity formed in the bone structure, leaving only the guide pin in the cavity. The striated screw is then placed in the bone structure by the guide pin.

In this example, the detachable part includes only the internal electrode (3). It is understood that, according to another configuration, the detachable longitudinal part may comprise, in addition to the internal electrode (3), the insulant (4).

According to another configuration, the detachable longitudinal part forms the lateral periphery of the penetrating part (1) of the penetration instrument. Advantageously, the detachable longitudinal part includes the external electrode (2) and the insulant (4) (FIG. 3).

Thus, when the detachable longitudinal part is withdrawn, only the electrode (3) remains in the cavity (7) formed in the bone structure and forms the guide core (6). The guide core (6) formed by the electrode (3) is used as a guide, for example, to insert a striated screw.

As above, due to the arrangement of the electrodes (2, 3) in the penetration instrument (1), the guide core (6) is arranged centrally in the bone cavity. In this example, the guide core includes only the internal electrode (3). It is understood that, according to another configuration, the detachable longitudinal part may only involve the external electrode (2). In these conditions, the guide core will include the insulant (4) and the electrode (3).

This disclosure is described above by way of example. It is understood that a person skilled in the art is able to make different variations without departing from the spirit of the disclosure as defined in the appended claims.

1-10. (canceled)

11. A device to monitor penetration of an instrument into an anatomical structure comprising:
a voltage source supplying at least two electrodes, at least one of the electrodes being carried by a part of the penetration instrument that penetrates the anatomical structure, the penetrating part having at least one detachable longitudinal part; and
a means to measure the impedance between the electrodes or an electro-stimulator or both.

12. The device according to claim 11, wherein the penetrating part further comprises an operator channel comprising the detachable longitudinal part.

13. The device according to claim 12, wherein the operator channel comprises a detachable electrode.

14. The device according to claim 12, wherein the device further comprises a flexible or rigid guide pin, crossing the operator channel.

15. The device according to claim 12, further comprising a fluid injection tube crossing the operator channel.

16. The device according to claim 11, wherein the detachable longitudinal part is centrally positioned.

17. The device according to claim 11, wherein the detachable longitudinal part forms a lateral periphery of the penetrating part so that, when the longitudinal part is withdrawn, the penetrating part has a guide core.

18. The device according to claim 17, wherein the guide core comprises an electrode.

19. The device according to claim 17, wherein the guide core is centrally positioned.

20. The device according to claim 11, wherein the detachable longitudinal part is an electrode.

21. A device to monitor penetration of an instrument into an anatomical structure comprising:
a voltage source supplying at least two electrodes, at least one of the electrodes carried by a part of the penetration instrument intended to penetrate the anatomical structure, the penetrating part having at least one detachable longitudinal part; and
a device that measures the impedance between the electrodes or an electro-stimulator or both.
